(19) United States
(12) Patent Application Publication
(10) Pub. No.: US 2007/0123700 A1
(43) Pub. Date: May 31, 2007

(54) Processes for removing cells and cell debris from tissue and tissue constructs used in transplantation and tissue reconstruction

(76) Inventors: Yuichiro Ueda, Garden Grove, CA (US); Mark W. Torrianni, San Juan Capistrano, CA (US)

Correspondence Address: Katharine A. Jackson Huebsch;Medtronic, Inc., 710 Medtronic ParkWay, Minneapolis, MN, 55432, US

(21) Appl. No.: 11/698,818

(22) Filed: Jan. 26, 2007

Related U.S. Application Data
(62) Division of application No. 10/858,174, filed on Jun. 1, 2004, now abandoned.

(51) Int. Cl. C07K 1/10

(52) U.S. Cl. 530/412

(57) ABSTRACT

Methods for decellularizing mammalian tissue for use in transplantation and tissue engineering. The invention includes methods for simultaneous application of an ionic detergent and a nonionic detergent for a long time period, which may exceed five days. One method utilizes SDS as the ionic detergent and Triton-X 100 as the nonionic detergent. A long rinse step follows, which may also exceed five days in length. This long duration, simultaneous extraction with two detergents produced tissue showing stress-strain curves and DSC data similar to that of fresh, unprocessed tissue. The processed tissue is largely devoid of cells, has the underlying structure essentially intact, and also shows a significantly improved inflammatory response relative to fresh tissue, even without glutaraldehyde fixation. Significantly reduced in situ calcification has also been demonstrated relative to glutaraldehyde fixed tissue. Applicants believe the ionic and non-ionic detergents may act synergistically to bind protein to the ionic detergent and may remove an ionic detergent-protein complex from the tissue using the non-ionic detergent. The present methods find one exemplary use in decellularizing porcine heart valve leaflet and wall tissue for use in transplantation.

### CROSS-REFERENCE TO RELATED APPLICATIONS

The present application is a divisional application of U.S. patent application Ser. No. 10/858,174, filed Jun. 1, 2004, which is specifically incorporated by reference herein, and is related to U.S. Pat. No. 6,509,145.

### FIELD OF THE INVENTION

The present invention is related generally to implantable medical prostheses. More specifically, the present invention is related to bioprostheses made from tissue and tissue constructs. The present invention finds one (non-limiting) use in preparing mammalian tissue for use in making bioprosthetic heart valves.

### BACKGROUND OF THE INVENTION

The surgical implantation of prosthetic devices (prostheses) into humans and other mammals has been carried out with increasing frequency. Such prostheses include, by way of illustration, heart valves, vascular grafts, vein grafts, urinary bladders, heart bladders, left ventricular-assist devices, and the like. The prostheses may be constructed from natural tissues, inorganic materials, synthetic polymers, or combinations thereof. By way of illustration, mechanical heart valve prostheses typically are composed of rigid materials, such as polymers, carbon-based materials, and metals. Valvular bioprostheses, on the other hand, typically are fabricated from either porcine aortic valves or bovine pericardium.

Prostheses derived from natural tissues are preferred over mechanical devices because of certain clinical advantages. For example, tissue-derived prostheses generally do not require routine anticoagulation. Moreover, when tissue-derived prostheses fail, they usually exhibit a gradual deterioration that can extend over a period of months or even years. Mechanical devices, on the other hand, typically undergo catastrophic failure.

Although any prosthetic device can fail because of mineralization, such as calcification, this cause of prosthesis degeneration is especially significant in tissue-derived prostheses. Indeed, calcification has been stated to account for 50 percent of failures of cardiac bioprosthetic valve implants in children within 4 years of implantation. In adults, this phenomenon occurs in approximately 20 percent of failures within 10 years of implantation. See, for example, Schoen et al., J. Lab. Invest., 52, 523-532 (1985). Despite the clinical importance of the problem, the pathogenesis of calcification is not completely understood. Moreover, there apparently is no effective therapy known at the present time.

The origin of mineralization, and calcification in particular, has, for example, been shown to begin primarily with cell debris present in the tissue matrices of bioprosthetic heart valves, both of pericardial and aortic root origin. Bioprosthetic cross-linked tissue calcification has also been linked to the presence of alkaline phosphatase that is associated with cell debris and its possible accumulation within implanted tissue from the blood. Still others have suggested that mineralization is a result of phospholipids in the cell debris that sequester calcium and form the nucleation site of apatite (calcium phosphate). Others have suggested that elastin and its fibrillin subunits may be the nidus for calcification, because of the calcium binding capabilities of these proteins.

Regardless of the mechanism by which mineralization in bioprostheses occurs, mineralization, and especially calcification, is the most frequent cause of the clinical failure of bioprosthetic heart valves fabricated from porcine aortic valves or bovine pericardium. Human aortic homograft implants have also been observed to undergo pathologic calcification involving both the valvular tissue as well as the adjacent aortic wall albeit at a slower rate than the bioprosthetic heart valves. Pathologic calcification leading to valvular failure, in such forms as stenosis and/or regeneration, necessitates re-implantation. Therefore, the use of bioprosthetic heart valves and homografts has been limited because such tissue is subject to calcification. In fact, pediatric patients have been found to have an accelerated rate of calcification so that the use of bioprosthetic heart valves is contraindicated for this group.

Several possible methods to decrease or prevent bioprosthetic heart valve mineralization have been described in the literature since the problem was first identified. Generally, these methods involve treating the bioprosthetic valve with various substances prior to implantation. Among the substances reported to work are sulfated aliphatic alcohols, phosphate esters, amino diphosphonates, derivatives of carboxylic acid, and various surfactants. Nevertheless, none of these methods have proven completely successful in solving the problem of post-implantation mineralization.

Currently there are no bioprosthetic heart valves that are free from the potential to mineralize in vivo. Although there is a process employing amino oleic acid (AOA® (Biomedical Design, Inc.)) as an agent to prevent calcification in the leaflets of porcine aortic root tissue used as a bioprosthetic heart valve, AOA® has been shown to mitigate calcification in the leaflets of porcine bioprostheses, but has not been shown to be effective in preventing the mineralization of the aortic wall of such devices. As a result, such devices may have to be removed.

Currently available porcine heart valves can also cause varying degrees of immunogenic and inflammatory response. Current glutaraldehyde fixation methods significantly mask, but do not eliminate the antigenicity of the implanted porcine valve tissue. Porcine heart valves can cause an inflammatory response, ranging from mild to severe. In severe cases, the foreign tissue may cause a chronic inflammatory response. The inflammatory response may be due in part to the cytotoxic nature of glutaraldehyde itself.

Accordingly, there is a need for providing long-term calcification resistance for bioprosthetic heart valves and other tissue-derived implantable medical devices that are subject to in vivo pathologic calcification. There is also a need for methods providing xenogenic tissue having reduced inflammatory and immunogenic response.

### BRIEF SUMMARY OF THE INVENTION

The present invention includes methods for treating tissue to remove non-structural proteins from the tissue, making the tissue more suitable for transplantation. Tissue can include both excised mammalian tissue and tissue culture produced tissue constructs. Methods can include contacting the tissue with a first, ionic detergent and a second, non-ionic detergent. Applicants believe the ionic and non-ionic detergents may act synergistically to bind protein to the ionic detergent and may remove an ionic detergent-protein complex from the tissue using the non-ionic detergent.

The first detergent is an ionic detergent that is often capable of disrupting the cell membrane and binding protein. The first detergent is preferably an anionic detergent, for example sodium dodecyl sulfate or sodium dodecyl sulfonate. Bile salts, for example sodium cholate or sodium deoxycholate, may be used in an alternate embodiment of the invention.

The second detergent has a net neutral charge, and can be an anionic detergent or a zwitterionic detergent operating at a pH to produce a net neutral charge. Examples of anionic detergents include polyethylene glycol containing detergents, such a polyethylene glycol sorbitan monolaurate (available as Tween 20), and the polyoxyethylene p-t-octly phenols (available as Triton X-100 and IGEPAL CA-630, depending on chain length). The first and second detergents are both in contact with the tissue at the same time, in non-negligible concentrations. In various methods, the first and second detergents are in contact with the tissue for at least 2, 3, 4, or 5 days or for a time suitable to insudate/penetrate a given matrix depending on its composition and density.

The first and second detergents are both present in at least 0.1, 0.2 or 0.5 weight percent in various methods, and have a combined presence of at least about 0.5 weight percent in some methods.

The detergent contacting step is a decellularizing step, which includes rupturing the cell membranes. In this step, the detergents are allowed to diffuse deeply into the tissue. The detergent contacting step can be followed by a rinse step, which can have about the same time duration as the detergent contacting step. The rinse step can remove the cell debris, including non-structural proteins, nuclei, organelles, globular proteins, and other materials, along with the detergents and any complexes formed between the various detergents and cell debris. The rinse step can include rinsing with a protease inhibitor, and other compounds that inhibit proteases, for example EDTA, which inhibits metaloproteases by chelating divalent cations necessary for their function. The rinse step can also include use of an anti-microbial agent, for example, sodium azide.

The detergent contacting step can be preceded by a wash step, to remove loose tissue and blood. The wash step can occur under agitation, and can include contacting the tissue with a protease inhibition cocktail and chelating agents to inhibit some enzymes that would otherwise degrade the tissue.

In one method, porcine aortic root tissue is excised from an animal, and washed with a saline solution including a protease inhibitor cocktail, a chelating agent, and sodium azide, for a couple days, with agitation. The washed tissue can then be decellularized by contact for about 5 days, under agitation, with a saline solution including the anionic detergent sodium dodecyl sulfate (SDS), the non-anionic detergent Triton X-100, and the anti-microbial agent sodium azide.

In this method, the decellularized tissue can then be rinsed for about 5 days under agitation, with a saline solution including sodium azide. The rinsed tissue can be sterilized through contact for about 3 hours with a saline solution including a chelating agent, sodium azide, isopropyl alcohol, and CPC. The sterilized tissue can be stored in a buffered saline solution including a chelating agent, sodium azide, and HEPES.

The present invention includes tissue products produced using methods according to the present invention. The tissue produced using these methods can be essentially devoid of nuclei when observed using standard histological techniques. This lack of nuclei extends deeply into the tissue, for example, into the middle of dense, 2 ½ or 3 millimeter thick porcine aortic root tissue. In various embodiments, at least about 80%, 90%, and 95% of the original non-structural (non-collagen, non-elastin) proteins have been removed from the tissue. In various embodiments, at least about 80%, 90%, and 95% of the total extractable protein has been removed from the tissue. In various embodiments, at least 70 and 80% of the DNA has been removed from aortic wall tissue, and at least 80 and 90% of the DNA has been removed from the valve leaflet tissue. These removal percentages apply to an average taken across the tissue thickness, even for 2 or 3 millimeter thick tissue. These removal percentages also apply to samples taken from ½ millimeter or 1 millimeter depths, or at the center of the tissue thickness, for 2, 2 ½, or 3 millimeter thick tissue.

Various embodiments produce at least a 80%, 90%, or 95% removal rate of original nuclei, when evaluated by standard light microscopy. This removal rate is based on the observed lack of observed intact or pichnotic (shrunken) nuclei, both typically observed in the resulting tissue from other processes. Often histological studies have shown the presence of a diffusely staining (basophilic) material in the areas where cells once resided, this may be a remnant of the nucleus and represents a very small fraction of the total cell content. These nuclei removal percentages also apply to averages across the tissue, and to samples taken from ½ millimeter or 1 millimeter depths, or at the center of the tissue thickness, for 2, 2 ½, or 3 millimeter thick tissue.

This lack of observable nuclei, and the inferred lack of other non-structural cellular material, can provide an improved tissue for use in xenogenic transplantation.

The processed tissue can be largely devoid of cells, and has the underlying structure essentially intact. Tissue prepared using the present methods, shows almost no protein when the samples are run under SDS gel electrophoresis. Animal implant studies of tissue prepared using the present invention has shown a significantly improved inflammatory response relative to fresh tissue, and to glutaraldehyde fixed tissue controls. Tissue prepared according to the present invention has shown a significantly improved immunogenic response compared to fresh tissue. Significantly reduced in situ calcification has also been demonstrated relative to glutaraldehyde fixed tissue.

Products produced according to the present invention include aortic root tissue, aortic wall tissue, heart valve leaflet tissue, blood vessels, ureters, fallopian tubes, and tissue constructs derived from in vitro tissue engineering. The present invention may find use in dermal dressings, incontinence procedures, as surgical mesh, neural tubes, as tendons, in orthopedic procedures, and in bladder and vaginal reconstruction procedures.

### BRIEF DESCRIPTION OF THE DRAWINGS

FIG. 1 is a photomicrograph of H.E. stained fresh, unprocessed, leaflet and wall tissue from a porcine heart valve;

FIG. 2 is a photomicrograph of H.E. stained leaflet and wall tissue decellularized using long term, simultaneous extraction with SDS and Triton-X 100 followed by a long-term rinse (a “hybrid process”);

FIG. 3 is a photomicrograph of MOVAT stained fresh, unprocessed, leaflet and wall tissue from a porcine heart valve;

FIG. 4 is a photomicrograph of a MOVAT stained decellularized leaflet and wall tissue, showing the substantially intact structure;

FIG. 5 is a DSC summary chart showing the similar thermal melt temperatures of the hybrid process decellularized leaflet and the fresh leaflet;

FIG. 6 is an SDS-PAGE gel electrophoresis, showing the substantial reduction in extractable protein in tissue prepared according to the present invention;

FIG. 7 is a stress-strain curve for fresh and processed tissue;

FIG. 8 is a graph of tissue calcium amounts after 60 days in a rat subdermal implant, showing a substantial reduction in calcification when using the present invention;

FIG. 9 contains four photomicrographs of tissue in a rabbit subdermal implant, showing a substantial reduction in inflammation after decellularization according to the present invention;

FIG. 10 is a chart of total extractable protein in fresh and decellularized porcine valve wall and leaflet tissue;

FIGS. 11A-11F are chemical structures of various detergents used in some examples of the present invention; and

FIG. 12 is a chart showing the DNA content of fresh and decellularized aortic root wall tissue and leaflet tissue.

### DETAILED DESCRIPTION OF THE INVENTION

### Mammalian and Tissue Culture Sources

The present invention can provide a tissue derived implantable medical device. The device can utilize tissue obtained from a mammalian species. Mammalian species include, for example, porcine, bovine, ovine sheep, equine, and the like. Examples of tissue include, but are not limited to, porcine aortic root tissue, bovine aortic root tissue, facia, omentum, porcine and/or bovine pericardium or veins and arteries, including carotid veins and arteries. The tissue includes a heart valve in some examples of the invention. The present invention can also utilize tissue obtained as a tissue construct produced in vitro through cell culture.

### Tissue Sources

The tissue for such a tissue-derived implantable medical device can be obtained directly from a slaughterhouse, and be dissected at the slaughterhouse to remove undesired surrounding tissue. Either at the slaughterhouse or shortly thereafter, but prior to significant tissue damage and/or degradation, the tissue can be treated according to methods of the present invention. In one method, once the tissue is obtained, it is shipped on ice in order to reduce autolytic damage to the tissue and to minimize bacterial growth during shipment. In some methods, the tissue is shipped and received within about 24 hours to a location where treatment of the tissue, as described herein, can be performed.

In one embodiment of the invention, the tissue is placed in a detergent solution at the harvesting site. The tissue may be placed into such solution within two hours, one hour, or even a half-hour from the time of removal from the animal. This detergent solution can be one of the two detergent solutions described elsewhere with respect to the decellularization step. The decellularization step can thus begin immediately after slaughter, before tissue can begin autolytic degradation. When such immediate contact with detergent is performed, the wash step may be performed after the tissue is received at the tissue treatment site, followed by further contact with decellularization solution, or the decellularization solution can be changed with fresh decellularization solution, and the wash step effectively skipped.

### Wash Step

In one method, the tissue is thoroughly washed with a chelating, non-phosphate saline (NPS) solution. The solution may stabilize the tissue matrix while assisting in the removal of excess blood and body fluids that may come in contact with the tissue. This NPS solution can be used in the present invention, as applicants believe it serves to remove phosphate-containing material and reduce enzyme activity that requires divalent cations from the tissue derived implantable medical device. This is desirable as such enzyme activity can degrade the cellular matrix.

The chelating, non-phosphate saline solution, suitable for use in the present invention can contain additional components, for example, a saline solution, preferably 0.1% to 1.0% by weight. The chelating agent is present in the solution at a concentration of about 10 mM to about 30 mM in some embodiments. Suitable chelating agents include, for example, EDTA (ethylenediaminetetraacetic acid), EGTA (ethylenebis oxyethylenenitrilo teteraacetic acid), citric acid, or salts thereof, and sodium citrate. A chelating agent employed in some methods according to the present invention can bind divalent cations, such as calcium, magnesium, zinc, and manganese. Binding such ions can inactivate enzymes that utilize divalent cations. Removal of such ions from the tissue derived from the mammals may render the tissue less susceptible to spontaneous precipitation (apatite formation) of these divalent ions with phosphate ions that may be present in the tissue. Also, taking away divalent cations can inhibit a specific family of degradative enzymes, known as matrix metaloproteases (MMPs), from breaking down the matrices during treatment. An antibacterial compound, for example, sodium azide, may also be included in the wash solution.

Protease inhibition cocktails including, for example AEBSF (Available from Sigma as P2714) can also be used. Some cocktails include EDTA, AEBSF, E-64, B-statin, leupeptin, and aprotinin. As used herein, “protease inhibition cocktails” refer to something more than “protease inhibitors”, which may contain only chelating agents as EDTA. Protease inhibition cocktails are significantly more expensive than divalent ion chelators used in the inhibition of metaloproteases .

In one embodiment, the chelating, non-phosphate solution of the invention is about 0.3% (w/v) saline, has a pH of about 7.4, contains about 10 mM to about 20 mM of EDTA, and about 0.05% wt/vol sodium azide. Subsequent to rinsing in the chelating, non-phosphate saline solution, as described above, the derived tissue may be maintained at about room temperature from about 24 hours to about 48 hours, until further processing.

Regardless of the specific wash treatment protocol employed, the non-phosphate saline solution to tissue ratio, i.e. the volume to tissue ratio, is preferably fairly large in one embodiment of the invention. Applicants believe that a large volume to tissue ratio maintains a high concentration gradient for solute diffusion from the tissue (and from the tissue extracellular matrix), away from the tissue and out into the surrounding chelating, non-phosphate saline solution. Some methods utilize at least about 15 ml of solution per gram of wet weight tissue. Frequent volume changes can aid in maintaining the diffusion gradients to assist in removal of compounds from the ECM. During the wash treatments of the present invention, the tissue can also be subject to mechanical processing by any number of methods. In one such method, a roller bottle apparatus can be employed to keep the treated tissue suspended in the extraction bed volume during treatment. Employing a tissue roller bottle apparatus may be advantageous in that it may further assist in the diffusion of materials from the tissue by maintaining the concentration gradient between materials to be extracted from the matrix and the concentration of the material in the volume of the extraction solution. The temperature during the wash treatment can be maintained at about room temperature, for example, between about 20 degrees C. and 30 degrees C.

Applicants believe that the washing step allows for the removal of extraneous tissue debris, blood components, and the inhibition of matrix metaloproteases through action of the chelating agents.

### Decellularization Step

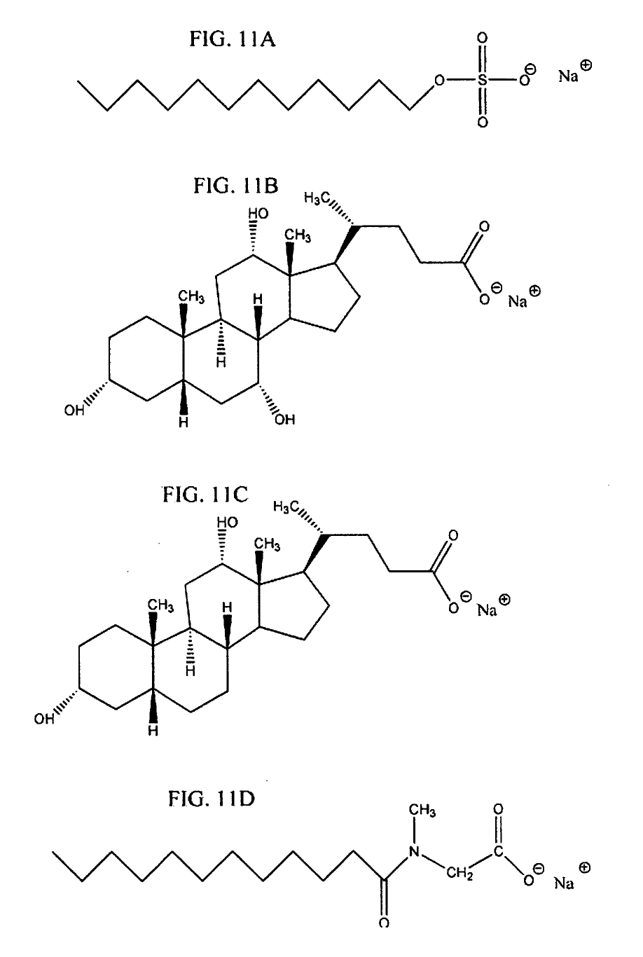

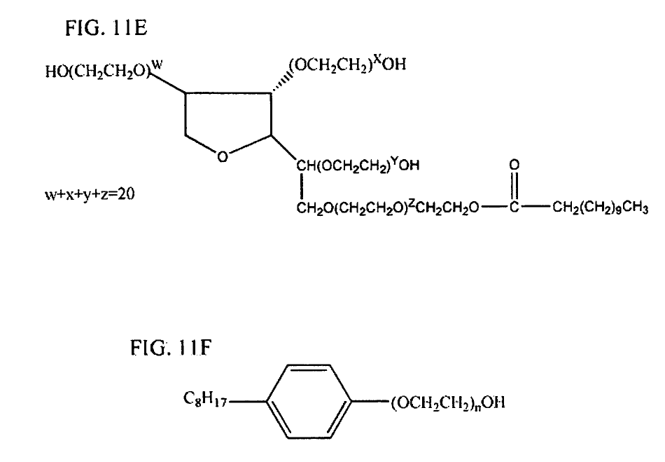

After the tissue has been washed, the tissue can be brought into contact with the decellularization solution. The solution contains at least two different detergents. One detergent is preferably non-ionic and the other is preferably anionic. Non-ionic detergents include those available as Triton X-100, NonidetP-40 (NP-40), IGEPAL CA-630, and Tween 20. Detergents are discussed further in the text associated with FIGS. 11A-11F. Anionic detergents include Sodium Dodecyl Sulfate (SDS), Sodium Dodecyl Sulfonate, and Sodium Dodecyl Sarcosinate.

The tissue can be brought into contact with the two detergents by mixing the two detergents and bringing the tissue into contact with the mixture, or adding one detergent, then the other, to the tissue. The present invention includes methods for bringing the tissue into contact with the at least two detergents for a time period, which could include adding one of the detergents first for an initial time period before adding the second detergent to begin the period of simultaneous contact with the multiple detergents.

The concentrations of the detergents can be varied depending on the desired extraction requirements. The total concentration of all detergents added to the extraction mixture does not exceed about 2 weight percent in some embodiments. The total concentration of detergents in some methods is between about 0.25 percent and about 2 percent (weight by volume for solid detergents, or volume by volume for liquid detergents). The total concentration of detergents in other methods is between about 0.5 percent and about 1 percent (weight by volume for solid detergents, or volume by volume for liquid detergents).

The detergent composition can also contain between about 0.1% and 1% saline by weight and between about 0.025% and 0.1% by weight sodium azide. The tissue can be placed in the detergent containing composition for periods of at least about 3, 4, or 5 days, or from 2 days up to 5 days, depending on the embodiment and depending on the tissue thickness, and/or tissue density. The tissue-contacting period can be long enough to insudate the center of the tissue thickness, with the subsequent rinse step being long enough to remove most of the detergent from the tissue thickness center. The tissue and detergents can be maintained at a temperature of between about 20 to 40 degrees C. in some methods. In one method, the tissue is placed in contact with the detergents for at least about 5 days. In another method, pericardium about ¼ mm thick is placed in contact with the detergents for about 2 days. Tissue constructs from tissue culture may require only about 2 days, or less, depending on the construct.

The tissue constructs may be used as implants, tissue fillers, burn dressings, wound dressings, and other applications well known to those skilled in the art. Blood vessels, such as mammalian (e.g. porcine or bovine) veins or arteries may be cleansed of native protein and used for blood vessel grafts or replacements in humans.

Applicants believe the detergents facilitate the breakdown of cellular structure for the removal of cells as well as cell debris, and cell organelles from the ECM. This includes significant removal of protein. The detergent treatment breaks up the phospholipid bilayer of cell membranes in the process of extracting the proteins. While not wishing to be bound by theory, applicants believe that the ionic detergent may disrupt the cell membrane and also bind to the protein forming an ionic detergent-protein complex. The non-ionic detergent may then solubilize the ionic detergent-protein complex and/or perhaps some proteins, and may assist in removing this complex from the tissue. The solubilization may also reduce the precipitation of protein in the tissue and/or ionic detergent -protein complexes in the tissue, assisting in their removal during the rinse step. Denser tissue may require longer contact times. For example, femoral artery tissue may require a longer contact time than femoral vein tissue.

One ionic detergent is an anionic detergent. In particular, sodium dodecyl sulfate (SDS) and SDS derivatives can be used as anionic detergents. Some embodiments of the invention use sodium dodecylsulphonate or sodium dodecyl-N-sarcosinate and derivatives thereof.

Some non-ionic detergents have polyoxyethylene chains and aliphatic chains. The present invention includes non-ionic detergents, for example: polyoxyethylene p-t-octyl phenol (available under tradenames Triton X and IGEPAL); polyoxyethylene sorbitol esters (available under the tradenames Tween and Emasol); polyoxyethylene alcohols (available under the tradenames Brij, Lubrol W and Lubrol AL); polyoxyethylene isoalcohol (available under the tradenames Sterox AJ and Sterox AP, Emuphogen BC, and Renex 30); polyoxyethylene nonyphenol (available under the tradenames Triton N, IGEPAL CO, and Surfonic N); and polyoxyethylene esters of fatty acids (available under the tradenames Sterox CO, Myrj, and Span). Zwittergens (Zwitterionic detergents) are used in other embodiments in place of or in addition to the non-ionic detergents, at pH appropriate to provide a net neutral charge.

FIGS. 11A-11F contain chemical structures of detergents used in some embodiments of the present invention. FIG. 11A illustrates sodium dodecyl sulfate (SDS). Sodium dodecyl sulfonate, used as an ionic detergent in some embodiments, has the sulfur directly bonded to the alphatic chain. Alkyl sulfates and sulfonates are used as ionic detergents in some methods according to the present invention. FIG. 11B illustrates sodium cholate, another ionic detergent. FIG. 11C illustrates sodium deoxycholate (DOC), yet another ionic detergent. FIG. 11D illustrates N-lauroylsarcosine sodium salt. Adding one more carbon to the aliphatic chain would provide another ionic detergent used in some embodiments of the present invention. Sodium dodecyl sarcosinate is used in some embodiments of the invention. FIG. 11E illustrates polyoxyethylene sorbitan monolaurate, having the indicated structure, where the sum of w, x, y and z is equal to 20. This detergent is an anionic detergent available under the trade name Tween 20. FIG. 11F illustrates a family of anionic detergents, where n varies, having values ranging from 8 to 12 in some embodiments. When n is about 8 (on average), the detergent may be referred to as nonylphenyl-polyethylenglycol, (octylphenoxy) polyethoxyethanol, or octylphenyl-polyethylene glycol, available under the trade names Nonidet P 40, NP-40, or IGEPAL CA 630. When n is equal to about 10 (on average), the detergent may be referred to as (decylphenoxy) polyethoxyethanol, or decylphenyl-polyethylene glycol, available under the trade name Triton X-100.

### Rinse Step

After treatment with the detergents composition, the tissue can be further processed through exhaustive rinse processes utilizing non-phosphate saline solution. The content of the rinse solution can be between 0.1 and 1.0 weight percent saline and between about 0.025 and 0.12 weight percent sodium azide. Protease inhibitors such as EDTA, EGTA, and sodium citrate-citric acid can also be added. The tissue can be placed in the rinse solution for a period of at least 3, 4, or 5 days, in various embodiments. The rinse solution temperature may be maintained between about 20 and 40 degrees C. in some methods. In one method, the tissue is placed in contact with the rinse solution or solutions for at least about 5 days. This rinse step is about the same length of time, or at least the same length of time, as the decellularization step in some embodiment methods. The rinse step can be long enough to remove most of the detergents, from the tissue center.

### Sterilization Step

After the rinse step, the tissue can be processed through a sterilization step by contacting the tissue with a sterilization solution. The solution can contain between about 0.1 and 1.0 weight percent saline, about 10 mM to 30 mM EDTA, about 0.5% to about 5.0% (by volume) Isopropyl alcohol, and about 0.1% to 0.25% (by weight) Cetylpyridinium chloride (CPC). Tissue can be left in contact with the sterilization solution for 1 hour to 3 hours in some embodiments, and about 3 hours in one embodiment.

### Storage

After sterilization, the tissue can be packaged in storage solution. In one method, the contents of the storage solution can contain about 0.1% to about 1.0% (by weight) saline, about 10 mM to about 30 mM EDTA, about 5 mM to about 20 mM HEPES at pH 7.4, and about 0.01% to 0.1% (by weight) sodium azide. The tissue may be stored at temperatures between about 10° C. to 40° C. until use.

The invention will now be described by the following non-limiting Examples:

### EXAMPLES

In an exemplary embodiment of the invention, several types of tissue were evaluated; porcine aortic root tissue (PART) that has applications in heart valve replacement surgery, veins, facia, and pericardial tissue (PT), which can be used in either cardiovascular applications or as a general tissue support throughout the body.

### Wash step

Porcine Aortic Root Tissue (PART) and Pericardial Tissue (PT) were brought into the lab and washed in a wash solution comprising: 0.3% Sodium chloride; 20 mM EDTA; protease inhibitor cocktail as previously described; and 0.05% sodium azide.

The tissue was dissected free of unwanted connective and adipose tissue and placed back in a fresh wash solution prior to washing. The wash step was carried out in 2-liter tissue culture bottles, placed on a roller apparatus designed to rotate the bottles at 60 RPM for 24 hours at room temperature. This process also assisted in the removal of unwanted tissue and debris from the tissue.

### Decellularization Step

After rinsing, the wash solution was decanted off the tissue and the volume was replaced with a solution for decellularizing the ECM. The solution contained the following: 0.3% Sodium chloride; 0.5% Sodium laurel sulfate (a.k.a. sodium dodecyl sulfate); 0.5% Triton-X 100; and 0.05% Sodium azide.

The valves, having wall tissue from about 1 ½ to 2 mm in thickness, were exposed to the decellularization solution for about 144 hours +/−24 hours, at room temperature, while being rotated in the bottles at 60 RPM. The solution had a hypotonic character, with an osmolality of 120 to 130 mOsm/kg. Applicants have found that using a combination of detergents in conjunction with hypotonicity facilitates disruption of cells within the tissue ECM, without altering the major structural components of the matrix such as collagen and elastin.

### Rinse Step

After exposure to the decellularization solution, the solution was decanted off the tissue and placed in a rinse solution containing: 0.3% Sodium chloride; and 0.05% Sodium azide.

The rinse solution was replaced frequently during the rinsing process, which was performed for 144 hours in this example, at room temperature. This solution was also hypotonic in nature, facilitating better removal of the cell debris from the ECM during the duration of the rinsing process.

### Sterilization Step

After the rinsing process, the tissue was sterilized by a “cold chemical treatment” comprising the following solution: 0.3% sodium chloride; 20 mM EDTA; 1.0% isopropyl alcohol; and 0.25% CPC. The sterilization process took place for 3 hours and was conducted at room temperature. The present invention explicitly includes using CPC in a tissue sterilization step, where the CPC may be used in conjunction with a chelating agent, for example, EDTA.

### Storage

Upon the completion of the sterilization process the tissue was aseptically transferred to a storage solution composed of the following: 0.6% sodium chloride; 20 mM EDTA; 10 mM HEPES; and 0.05% sodium azide. The tissue was stored, in various runs, at a temperature of about varying between about 4 and 40 degrees C.

### EXPERIMENTAL RESULTS

### Structural Analysis

Tissue morphology/structural integrity of tissue processed by the above exemplary embodiment of the invention (decellularized tissue) was assessed by histology, transmission electron microscopy (TEM), and differential scanning calorimetry (DSC). A variety of staining procedures were employed to assess the structure and presence of various components of the PART and PT. H.E. staining was used to show overall tissue morphology and is the preferred stain in many pathology evaluations. MOVAT staining was used to show the distribution of various components of the tissues ECM, especially collagen, elastin and GAGs.

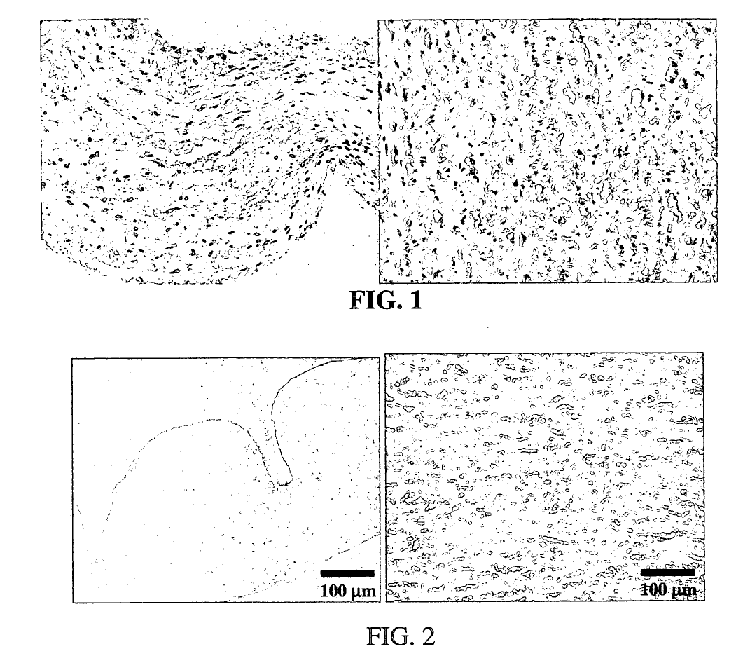

FIG. 1 shows a photomicrograph of H.E. stained fresh porcine heart valve tissue. The fresh porcine tissue is unprocessed, with the leaflet tissue being shown on the left and the wall tissue being shown on the right. The distribution of cells within the leaflet and the wall extracellular matrix may be seen, as the cell nuclei show up as black in FIG. 1. The presence of the nuclei, together with the cell membrane, associated organelles, and proteins may be inferred from viewing FIG. 1. The organized nature of the leaflet and wall tissue structure may also be seen in FIG. 1.

FIG. 2 is a photomicrograph of H.E. stained decellularized processed leaflet and wall tissue. The tissue was decellularized using “a hybrid” process according to the present invention. Specifically, this included an extraction step using a hypotonic solution containing 0.5% SDS and 0.5% Triton-X 100 simultaneously with agitation for a period of 144 hours at 25° C., followed by an extensive rinse step, also of 144 hours in length. About 500 ml of rinse solution was used per valve per rinse. The rinse was repeated with fresh solution. It can be seen that virtually all nuclei have been removed, and the attendant removal of membranes, cytoplasm, proteins, and cellular organelles may be inferred. Upon close observation of FIG. 2, it can be seen that the tissue structure is maintained similar to the original organization seen in FIG. 1. Applicants have used such histological screening as a first step in evaluating the efficacy of the decellularization processes.

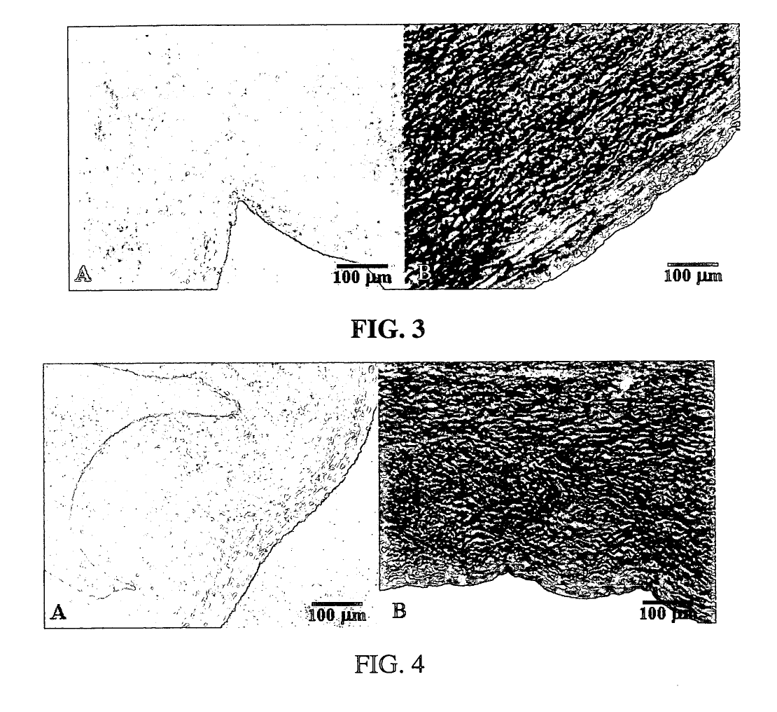

FIG. 3 is a photomicrograph of MOVAT stained fresh, unprocessed porcine heart valve tissue, with leaflet tissue being shown at the left at A and denser wall tissue being shown at the right at B. In the original color photo, nuclei are red, elastin fibers may be seen in dark purple, collagen and reticular fibers in yellow, ground substance and mucin in blue, fibrinoid and fibrin in intense red, and muscle in red.

FIG. 4 illustrates MOVAT stained decellularized porcine heart valve tissue, using the hybrid process according to the present invention, described with respect to FIG. 2. The decellularized leaflet may be seen at the left at A, and the wall tissue at the right at B. In the original, color photograph, the same color staining may be seen as in FIG. 3. FIG. 4 illustrates that the components are in their proper orientation, as in FIG. 3. In particular, FIGS. 3 and 4 show that the structural integrity of the heart valve tissue is maintained after the decellularization process. The structure of the heart valve tissue appears to resemble that of the fresh, unprocessed tissue.

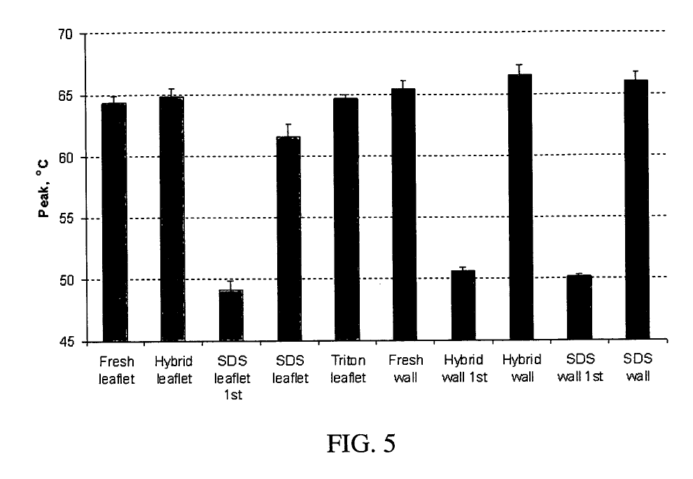

FIG. 5 illustrates Differential Scanning Calorimetry (DSC) data, having thermal melting point data for various tissues. The thermal melt point of type I collagen is between about 64 and 66 degrees Centigrade. This may be seen as indicated at “fresh leaflet” in FIG. 5. The thermal melt point is believed by applicants to reflect the change or lack of change in the tertiary or quaternary structure of the collagen in the tissue. Applicants believe that an unchanged thermal melt point is likely indicative of a substantially unchanged tertiary or quaternary collagen structure.

FIG. 5 also shows the thermal melt results for a porcine leaflet extracted using a hybrid process according to the present invention. As may be seen from FIG. 5, the thermal melt temperature is substantially the same, and within the error bar, of that of the fresh leaflet. Applicants believe that this indicates that the collagen structure is not substantially changed or damaged by the hybrid process. Next, tissue extracted for a long time period with SDS is shown to have two peaks, indicated at “SDS leaflet first” and “SDS leaflet”, respectively. The first SDS thermal melt peak may be indicative of residual SDS in the matrix interfering or intercollating into the collagen triple helix, causing it to melt at a lower temperature than normal. The second SDS leaflet peak may be seen to be closer to the fresh leaflet thermal melt point, but still significantly changed relative to the fresh leaflet thermal melt point. The thermal melt temperature for a Triton-X 100 treated leaflet may also be seen, indicated at “Triton leaflet.” Applicants believe that this represents that the Triton-X 100 alone is not effective in breaking down cell membranes and extracting protein.

The fresh wall tissue thermal melt may be seen as indicated at “fresh wall”, with the hybrid process yielding both a first and a second peak, at “hybrid wall first” and “hybrid wall”, respectively. Applicants believe that the hybrid wall first peak may be indicative of some residual SDS, and note that the hybrid wall second peak is very close to the thermal melt point of the fresh wall. Extensive rinse experiments support this residual SDS first peak hypothesis. If the material is rinsed extensively, the first peak goes away, leaving the primary peak for Type I collagen. The SDS alone results may be seen at a second and first peak, indicated at “SDS wall first” and “SDS wall”, respectively. Applicants believe that FIG. 5 illustrates that the collagen structure of this hybrid process treated tissue is not substantially different from that of the fresh, unprocessed tissue.

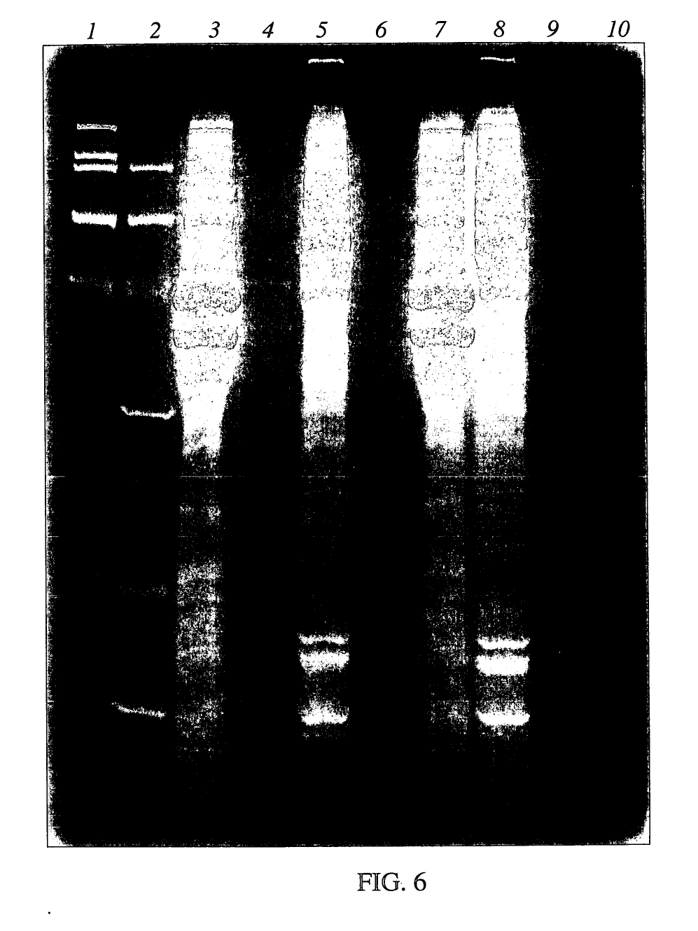

FIG. 6 illustrates an SDS gel electrophoresis page showing a substantial reduction in residual protein in tissue prepared using a hybrid process according to the present invention. Tissue was prepared by taking 50 micrometers thick cryo sections, and using 40 sliced wall tissues. The protein was extracted by grinding the cryo section slices into powder and extracting with 10 milliliters of 1% SDS solution. 20 microliters of the solution was deposited in each well. Lane 1 contains a high molecular weight standard. Lane 2 contains a low molecular weight standard. Lane 3 contains the fresh wall sample, showing a large amount of protein. Lane 4 shows only a small amount of extractable protein remaining in the tissue processed using the hybrid process. In particular, only three faint bands may be seen in the original, at approximately the position of the three brightest bands in the fresh wall sample in Lane 3.

Lane 5 contains the fresh leaflet sample, while Lane 6 contains the decellularized leaflet tissue, processed using the hybrid process. Lane 6 does not have even the faint bands of Lane 4. The material used in Lanes 4, 6, 9 and 10 was concentrated relative to that of Lanes 3, 5, 7, and 8. If not concentrated, almost nothing would be seen. Lanes 7, 9, 8 and 10 duplicate the samples of Lanes 3, 4, 5, and 6; respectively. These show the same respective results.

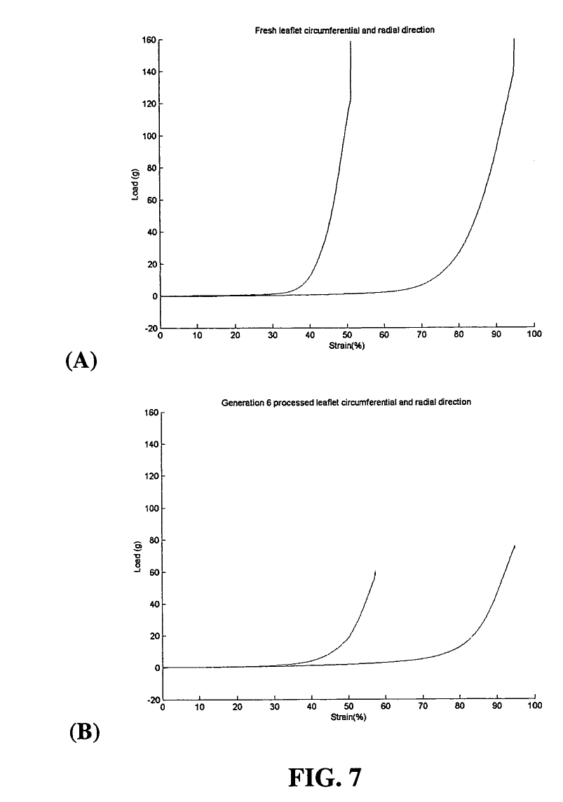

FIG. 7 illustrates stress-strain curves of fresh tissue at A and hybrid aortic root leaflet tissue at B. In each graph, the left curve is taken in the circumferential direction, while the right curve represents the radial direction. The stress-strain curves were generated to determine again whether the tissue treated using the hybrid process retains its structural integrity, relative to fresh tissue. FIG. 7 illustrates that tissue treated with the decellularization process yields curves that are closely matched to that of fresh leaflets. Applicants believe that this indicates that normal mechanical properties have been preserved.

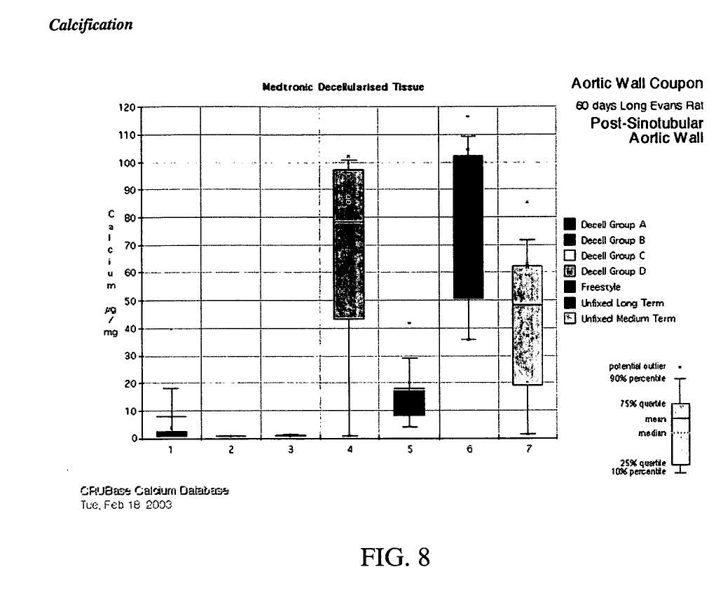

FIG. 8 is a plot of resulting rat subdermal implant calcification using different tissue treatment processes. Tissues treated in various processes were implanted for 60 days in long Evans rats. The calcium amount after 60 days was determined. In Lane 1, the calcification after long-term extraction with only SDS may be seen to be rather low. In Lane 2, the calcification in tissue treated using the hybrid process is extremely low. Lane 2 contains tissue treated long term with SDS and Triton-X 100, simultaneously, for an extraction and rinse period of about 144 hours. Lane 3 shows the calcification results for a hybrid process using SDS and IGEPAL, where IGEPAL is another nonionic detergent. The calcification may also seem to be very low. Lane 4 shows the calcification of tissue decellularized using the hybrid process, before glutaraldehyde fixation. The glutaraldehyde fixation may be seen to significantly increase the calcification. The reduction in calcification between glutaraldehyde fixed tissue and tissue treated only with decellularization may be seen by comparing Lane 4 to Lane 2.

Lane 5 shows the calcification results for a Freestyle® stentless valve treated with the AOA (amino oleic acid) followed by glutaraldehyde fixation. Lane 6 shows decalcification results for unfixed tissue that has been rinsed in saline and stored in saline at 4 degrees C. for a long time period in HEPPES at pH 7.4 with a chelating agent (EDTA) and 0.05 wt % sodium azide. Lane 7 shows the calcification results for unfixed tissue that has been rinsed to remove blood and debris and stored for a medium period of time before implantation.

Applicants believe tissue according to the present invention can maintain at least the structural integrity of glutaraldehyde fixed tissue, together with at least the immunogenic properties of glutaraldehyde fixed tissue, and at least the inflammatory response of glutaraldehyde fixed tissue. The reduction in calcification possible using decellularized tissue according to the present invention (without glutaraldehyde fixation), may be seen again by comparing Lane 4 to Lane 2.

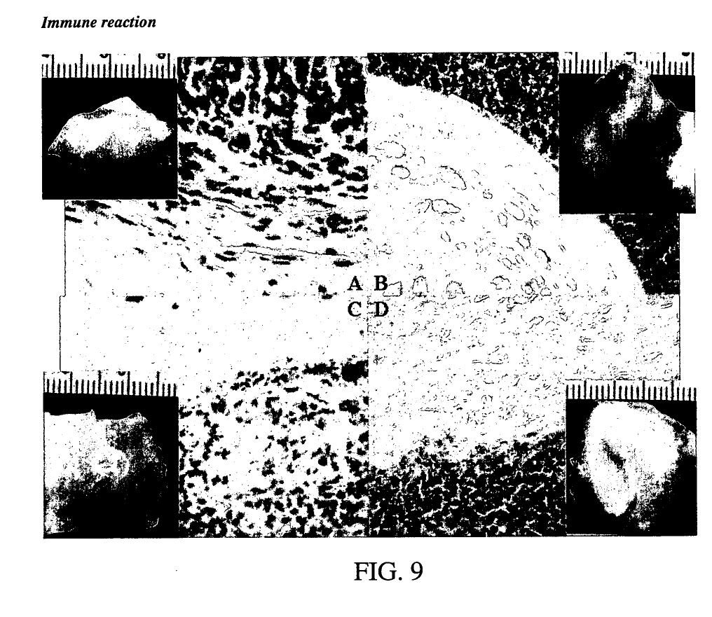

FIG. 9 illustrates the results of rabbit subdermal implants using various tissues. Tissues treated according to various methods were implanted subdermally in a rabbit. FIG. 9 shows the inflammatory reaction after two weeks. The results for a fresh leaflet are indicated at A, with the darkly stained areas indicative of large nuclei, for example, macrophages and neutrophils. Leaflet decellularized using the hybrid process is illustrated at B, showing a significantly reduced inflammatory response. The inflammatory response of fresh porcine wall tissue may be seen at C, with the dark stains again indicative of substantial inflammatory response. The results for decellularized wall tissue using the hybrid process may be seen at D, again indicative of a substantially reduced inflammatory response relative to that for fresh tissue.

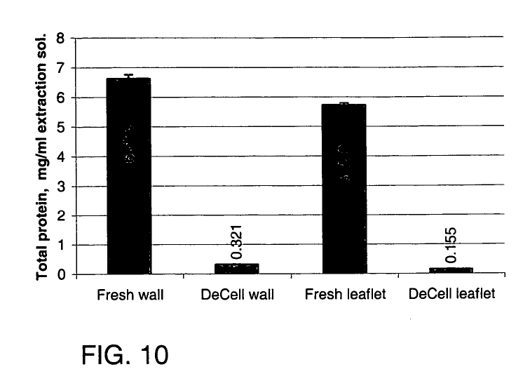

FIG. 10 shows yet another summary of data indicative of the successful protein removal using the present decellularization methods provided by the invention. Tissue treated according to the different methods was cryotomed, then crushed into dry powder. 0.3 grams of wall tissue was added to 10 milliliters of buffer of SDS running buffer, specifically, buffer containing SDS, glycine, and tris (available from Biorad as 161-0732).

Thus, the wall tissue solution contained 0.03 grams of wall dry powder per milliliter of buffer. For leaflets, 0.2 grams were added to 10 milliliters of SDS running buffer, producing a solution having 0.02 grams dry weight leaflet tissue per milliliter of buffer. The fresh wall tissue yielded 6.63 milligrams of total extractable protein per milliliter of extraction solution. The decellularized wall yielded only 0.321 milligrams total extractable protein per milliliter of extraction solution. Fresh leaflet yielded 5.73 milligrams extractable protein per milliliter of extraction solution while the decellularized leaflet yielded only 0.155 milligrams total extractable protein per milliliter of extraction solution.

For the fresh wall tissue, 6.6 milligrams per milliliter total protein was extracted from 0.03 grams dry weight tissue, or 221 milligrams total extractable protein per gram of dry weight tissue. This may be compared to only 10.6 milligrams total extracted protein per gram dry weight tissue for the decellularized wall of tissue. Similarly, the fresh leaflet contained 286.5 milligrams extractable protein per gram of dry weight protein compared to only 7.75 milligrams total extractable protein per gram of dry leaflet tissue.

Thus, there was a 95.2% reduction in total extractable protein when comparing the decellularized wall tissue to the fresh wall tissue. Similarly, there was a 97.3% reduction in total extractable protein when comparing the decellularized leaflet to the fresh leaflet. Applicants believe that this is a substantial reduction in protein relative to previous methods. As previously described, applicants believe that this substantial reduction in protein content of tissue may provide an improved immunogenic response, an improved inflammatory response, and a reduction in calcification.

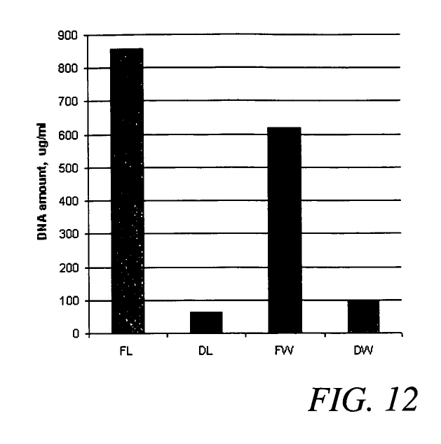

FIG. 12 illustrates another summary of data indicative of the removal of successful material removal from tissue using a method according to the present invention. The amount of DNA was measured in fresh leaflet tissue (FL), decellularized leaflet tissue (DL), fresh wall tissue (FW), and decellularized wall tissue (DW). Inspection and analysis of FIG. 12 shows that the decellularized leaflet tissue had about 93 percent of DNA removed relative to the fresh leaflet tissue, and the decellularized wall tissue had about 84% of the DNA removed relative to the fresh wall tissue. The present invention includes methods that remove at least about 80% and 90% of the DNA from heart valve leaflet tissue, and at least about 70% and 80% of the DNA from aortic root wall tissue.

### Cross-Linking And Decellularization

The present invention also includes methods for cross-linking tissue after decellularizing the tissue, and the resulting decellularized and cross-linked tissue. Reasons for possibly additionally desiring tissue crosslinking are now described. Host cells may move into the extra cellular matrix to remodel an implanted tissue matrix prepared using this methodology. Cellular movement into tightly constructed extra cellular matrices may require an active proteolytic system that cleaves a path for cell migration. During this time, when cells reestablish residence within the structures, the matrix structure may be compromised, in their biochemical function and strength. In some applications, for example, in fluid carrying blood vessels and in heart valves, the tissue strength may be beneficial during and after the remodeling. Therefore, it may be desirable to strengthen the tissue to better handle this period of relative weakness. The present invention can provide tissues decellularized using the methodologies described herein, and can also utilize tissue crosslinking methodologies to further treat and stabilize the matrices. The crosslinking can include both added length and zero length crosslinking.

Additionally, tissue may be decellularized at a point in the tissue preservation process, for example after using a process from U.S. Pat. No. 6,166,184, where primary amines have been blocked using a monofunctional aldehyde. In this example, the resulting Schiff base is reduced to a secondary amine by treatment with sodium cyanoborohydride. This blocked, yet uncrosslinked tissue may then be subjected to the present decellularization process.

In one example, tissue from a mammalian or tissue culture source can be decellularized using methods according to the present invention, followed by treatments described in U.S. Pat. No. 6,166,184, whereby primary amines of the decellularized tissue are blocked using a monofunctional aldehyde, followed by crosslinking of the matrix through a water soluble 1-ethyl-3-(3-dimethylaminopropyl) carbodiimide (EDC) methodology that utilizes a polypropyleneglycol spacer (available under the trade name Jeffamine from Texaco Chemical Company) as the di-amino bridge group to construct the crosslink.

In another example, tissue from a mammalian or tissue culture source may be treated with the monofunctional aldehyde of U.S. Pat. No. 6,166,184 initially, followed by the present application decellularization process, and then cross linked via the methodology also described in U.S. Pat. No. 6,166,184.

In another embodiment, tissue or tissue culture constructs decellularized by the current invention can be stabilized by treating the matrices with dilute solutions of buffered glutaraldehyde.

The matrices described herein can be further treated to enhance their biocompatibility by chemically attaching bioactive molecules such as cytokines, growth factors, anti-inflammatory compounds, anti-calcification compounds, non-thrombogenic substances, and heparin compounds.

Cross-linking tissue may be carried out using several types of methods. One group of methods utilize glutaraldehyde or other di-aldehydes to cross-link the tissue. Glutaraldehyde can react one or both aldehyde groups with amine groups on tissue protein to cross-link the tissue directly to other tissue or indirectly through polymers formed by the glutaraldehyde. See, for example, U.S. Pat. Nos. 3,966,401 and 4,050,893.

Another cross-linking method utilizes epoxy functionalized cross linking agents, which may be polyepoxy hydrophilic cross linking agents, polyol polyglycidylethers, and may be a diepoxide. Epoxy functionalized agents can include, but are not limited to, glycol diglycidyl ether, glycerol diglycidyl ether, glycerol triglycidyl ether and butanediol diglycidyl ether. Epoxy functionalized cross-linking agents can react with tissue carboxyl groups to cross-link the tissue. Unreacted tissue carboxyl groups may later be activated for cross-linking with tissue amines using an activating agent, which can include a carbodiimide. The tissue amines may be protected prior to the epoxy agent addition through use of a blocking agent. Examples of blocking agents include acylating agents and aminating agents. See, for example, U.S. Pat. No. 6,117,979.

Yet another crosslinking method includes: blocking at least a portion of the collagen amine groups with a blocking agent; activating at least a portion of the collagen carboxyl groups after blocking at least a portion of the collagen amine groups to form activated carboxyl groups; and contacting the activated collagen carboxyl groups with a polyfunctional spacer to crosslink the collagen-based material. The method may include the blocking agent being selected from the group consisting of an acylating agent, an aminating agent, and a biologically active derivative thereof. Blocking agents may include an acylating agent, for example, an N-hydroxy succinimide ester, a p-nitrophenyl ester, 1-acetylimidazole, and citraconic anhydride. The blocking agent may include an aminating agent, for example, an aldehyde or a ketone. Activating agents may include, for example, a carbodiimide, an azide, 1,1′-carbonyldiimidazole, N,N′-disuccinimidyl carbonate, 2-ethoxy-1-ethoxycarbonyl-1,2-dihydroquinoline, 1,2-benzisoxazol-3-yl-diphenyl phosphate, and N-ethyl-5-phenylisoxazolium-s′-sulfonate, and mixtures thereof. The carbodiimide can be water soluble. One example of a carbodiimide is 1-ethyl-3-(3-dimethylaminopropyl) carbodiimide (EDC) HCI.

The method step of reacting the activated collagen carboxyl groups with a polyfunctional spacer may include reacting the activated collagen carboxyl groups with a polyfunctional spacer and/or a diamine spacer. The diamine spacer may be hydrophilic, and may be selected from the group consisting of polyethyleneglycol spacers, polypropyleneglycol spacers, polyethylene-propyleneglycol spacers, and mixtures thereof. See, for example, U.S. Pat. No. 6,166,184.

In some methods, the decellularized tissue is treated with a first cross-linking agent containing either at least two reactive amino groups or at least two reactive carboxyl groups in the presence of the coupling agent, such that at least one of the reactive groups forms an amide bond with a reactive moiety on the tissue while another reactive group on at least some portion of the first cross-linking agent may remain free; and repeating the treatment described in the presence of the coupling agent with a second cross-linking agent containing at least two reactive carboxyl groups (if the first cross-linking agent used contains amino groups), or vice-versa if the first cross-linking agent contains carboxyl groups. Additional amide bonds are formed between reactive groups of the second cross-linking agent and either the free groups on the first cross-linking agent or reactive moieties on the tissue, resulting in the formation of links between or within the molecules of the tissue. Some of the links are chains containing at least one of both the first and second cross-linking agents. See, for example, U.S. Pat. No. 5,733,339.

Yet another cross-linking method includes treating the decellularized tissue with an effective amount of a coupling agent that promotes the formation of amide bonds between reactive carboxy moieties and reactive amino moieties in combination with a coupling enhancer, so as to result in the formation of amidated links to tissue reactive moieties. The method may also include treating the tissue with a cross-linking agent containing either at least two reactive amine moieties or at least two reactive carboxy moieties. The cross-linking agent may be a water-soluble di- or tri-amine or a water-soluble di- or tri-carboxylic acid, and the coupling agent may be water-soluble. The coupling agent may be a carbodiimide, for example, 1-ethyl-3 (3-dimethyl aminopropyl) carbodiimide (EDC). Where EDC is used, the coupling enhancer may be N-hydroxysulfosuccinimide (sulfo-NHS). See, for example, U.S. Pat. No. 5,447,536.

A decellularization process using the present invention can be applied at any point in a tissue stabilization process, where the tissue is not yet crosslinked, but where reactive −R groups have been previously modified (for example, as in U.S. Pat. No. 6,166,184), followed by decellularization, followed by additional −R group modification.

Thus, in one aspect, a specific −R group is modified using any type of chemical reaction scheme that does not crosslink the protein or molecule, followed by decellularization, followed by modification of a separate and unique −R group using an additional chemical reaction scheme, such that the tissue has modified −R groups, is decellularized, but not crosslinked. In another aspect, all potential reactive −R groups may be modified first, without crosslinking, followed by decellularization. In yet another aspect, the tissue can be decellularized, followed by −R group modification. In still another, prophetic aspect, the tissue is decellularized and modified during a single process.

In one example, the primary amines of proteins or other molecules (for example collagen) are blocked through the addition of a monofunctional aldehyde, the tissue is then decellularized, then other −R groups (for example carboxyl moieties) are modified by a water soluble EDC. In another example, carboxyls of proteins or other molecules are modified, followed by decellularization.

All publications, patents and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method of removing non-structural proteins from a tissue sample, comprising:
contacting the tissue with a first detergent capable of disrupting cell membranes in the tissue and binding to non-structural proteins in the tissue to provide a first detergent-protein complex and a second detergent having essentially a net neutral charge,
wherein the first and second detergents contact the tissue for a period of time sufficient to penetrate the tissue's center; and
rinsing the tissue to remove the first detergent, the second detergent, and non-structural proteins from the tissue.

2. The method of claim 1, wherein the second detergent is capable of forming a complex with the first detergent-protein complex so as to improve the solubility of the first detergent-protein complex.

3. The method of claim 1, wherein the tissue sample is mammalian.

4. The method of claim 3, wherein the sample is porcine.

5. The method of claim 3, wherein the sample comprises aortic root, aortic wall, heart valve leaflet, blood vessel, ureters, fallopian tubes, or tissue constructs derived from in vitro tissue engineering.

6. The method of claim 1, wherein contacting the tissue with the first and second detergents occurs simultaneously or sequentially.

7. The method of claim 6, wherein the contacting is simultaneous.

8. The method of claim 7, further comprising mixing together the first and second detergents prior to contacting the tissue to provide a decellularization solution, and contacting the tissue with the solution.

9. The method of claim 8, wherein the solution is hypertonic.

10. The method of claim 9, wherein the solution is in the range of about 120 mOsm/kg to about 130 mOsm/kg.

11. The method of claim 1, wherein the tissue is in the range of about ¼ mm to about 3 mm in thickness.

12. The method of claim 1, wherein at least about 80% of the non-structural proteins are removed from the tissue.

13. The method of claim 1, wherein the first detergent is an anionic detergent.

14. The method of claim 13, wherein the anionic detergent is sodium dodecyl sulfate, sodium dodecyl sulfonate, sodium dodecyl sarcosinate, a combination thereof or a derivative thereof.

15. The method of claim 1, wherein the second detergent is a non-ionic detergent.

16. The method of claim 15, wherein the non-ionic detergent is Triton X-100, Nonidet-P40 (NP-40), Igepal® CA-630, Tween 20, a combination thereof or a derivative thereof.

17. The method of claim 1, wherein the period of time is in the range of about 2 days to about 5 days.

18. The method of claim 1, further comprising agitating the tissue.

19. The method of claim 1, further comprising contacting the tissue with a sterilant selected from the group Cetylpyridinium chloride (CPC), CPC derivatives, and combinations thereof.

20. The method of claim 1, further comprising contacting the tissue with a chelating agent.
